(19) United States
(12) Patent Application Publication
(10) Pub. No.: US 2024/0188974 A1
(43) Pub. Date: Jun. 13, 2024

(54) MEDICAL DEVICE FOR ULTRASOUND-ASSISTED DRUG DELIVERY

(71) Applicant: Boston Scientific Scimed, Inc., Maple Grove, MN (US)

(72) Inventors: Benjamin Montag, Delano, MN (US); Joseph Czyscon, Elk River, MN (US); Christopher P. Knapp, Ham Lake, MN (US); Curtis Cornell Genstler, Snohomish, WA (US)

(73) Assignee: Boston Scientific Scimed, Inc., Maple Grove, MN (US)

(21) Appl. No.: 18/536,431

(22) Filed: Dec. 12, 2023

Related U.S. Application Data
(60) Provisional application No. 63/431,891, filed on Dec. 12, 2022.

(51) Int. Cl. A61B 17/22

(52) U.S. Cl. CPC A61B 17/22012; A61B 2017/00292; A61B 2017/22008; A61B 2217/007

(57) ABSTRACT

A system for treating a vascular region includes an elongate catheter shaft having a distal end region, with an inner lumen formed in the elongate shaft and a fluid delivery lumen formed in the elongate shaft adjacent to the inner lumen. A treatment core is disposable within the inner lumen, and has a plurality of ultrasound transducers that are adapted to move between a straight configuration and a helical configuration and to provide constructive interference when in the helical configuration.

### CROSS-REFERENCE TO RELATED APPLICATIONS

This application claims the benefit of priority under 35 U.S.C. § 119 of U.S. Provisional Application No. 63/431,891, filed Dec. 12, 2022, the entire disclosure of which is hereby incorporated by reference.

### TECHNICAL FIELD

The present disclosure pertains to elongated intracorporeal medical devices. More particularly, the present disclosure pertains to elongated intracorporeal medical devices that include ultrasound transducers.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices. Moreover, there is a need for medical devices that can provide constructive interference within ultrasound fields provided by ultrasound transducers in combination with introduction of lytic drugs.

### SUMMARY

This disclosure provides design, material, manufacturing method, and use alternatives for medical devices. An example may be found in a system for treating a vascular region. The system includes an elongate catheter shaft having a distal end region, the elongate catheter shaft defining an inner lumen extending through the elongate catheter shaft and a fluid delivery lumen extending adjacent to the inner lumen. The system includes a treatment core disposed within the inner lumen, the treatment core including a plurality of ultrasound transducers that are adapted to provide constructive interference.

Alternatively or additionally, the elongate catheter shaft may be adapted to move between a straight configuration and a helical configuration.

Alternatively or additionally, the treatment core may be adapted to move between a straight configuration and a helical configuration.

Alternatively or additionally, the fluid delivery lumen may have a distal end that is proximal of any of the plurality of ultrasound transducers.

Alternatively or additionally, the fluid delivery lumen may include a plurality of fluid delivery lumens, and at least some of the plurality of fluid delivery lumens may have a distal end that is offset from a distal end of others of the plurality of fluid delivery lumens.

Alternatively or additionally, the plurality of ultrasound transducers may have a treatment zone length when in the spiral configuration in a range of 6 centimeters to 50 centimeters.

Alternatively or additionally, the plurality of ultrasound transducers may have a spiral pitch when in the spiral configuration that is in a range of 3 centimeters to 6 centimeters.

Alternatively or additionally, the plurality of ultrasound transducers may have a spiral diameter when in the spiral configuration that is in a range of 6 millimeters to 30 millimeters.

Alternatively or additionally, each of the plurality of ultrasound transducers may be spaced 1 centimeter to 4 centimeters from adjacent ultrasound transducers when the plurality of ultrasound transducers are in the spiral configuration.

Another example may be found in an ultrasonic catheter system. The ultrasonic catheter system includes a multi-lumen catheter shaft having a central lumen and a plurality of fluid delivery lumens, and an ultrasound catheter core disposed within the central lumen, the ultrasound catheter core including a plurality of ultrasound transducers adapted to move between a straight configuration and a helical configuration. The plurality of ultrasound transducers are adapted to provide constructive interference when in the helical configuration. The central lumen is adapted to allow for a cooling media to pass therethrough when the ultrasound catheter core is disposed within the central lumen.

Alternatively or additionally, the multi-lumen catheter shaft may be adapted to move between a straight configuration and a helical configuration.

Alternatively or additionally, the ultrasound catheter core is adapted to move between a straight configuration and a helical configuration.

Alternatively or additionally, at least some of the plurality of fluid delivery lumens may have a distal end that is offset from a distal end of others of the plurality of fluid delivery lumens.

Alternatively or additionally, the plurality of ultrasonic transducers may be arranged for treating a PAO (peripheral arterial occlusion) disease state.

Alternatively or additionally, the plurality of ultrasonic transducers may be arranged for treating DVT (deep vein thrombosis).

Alternatively or additionally, the plurality of ultrasonic transducers may be arranged for treating PE (pulmonary embolisms).

Another example may be found in a system for treating a vascular region. The system includes an elongate catheter shaft having a distal end region, the elongate catheter shaft defining an inner lumen extending through the elongate catheter shaft and a fluid delivery lumen extending adjacent to the inner lumen. A treatment core is disposed within the inner lumen, the treatment core including two or more ultrasound radiating members, each of the two or more ultrasound radiating members adapted to provide constructive interference when in the helical configuration.

Alternatively or additionally, each of the two or more ultrasound radiating members may include a one-dimensional array of ultrasound transducers.

Alternatively or additionally, each of the two or more ultrasound radiating members may include a two dimensional array of ultrasound transducers.

Alternatively or additionally, one of the two or more ultrasound radiating members emit a radiated ultrasound field that overlaps with the radiated ultrasound field emitted by another of the two or more ultrasound radiating members.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of certain features of an illustrative ultrasonic catheter;

FIG. 2 is a cross-sectional view taken along line 2-2 of FIG. 1;

FIG. 3 is a schematic view of an illustrative elongate inner core configured to be positioned within the central lumen of the catheter shown in FIG. 2;

FIG. 4 is a cross-sectional view taken along line 4-4 of FIG. 3;

FIG. 5 is a schematic view of an illustrative elongate treatment core shown in a linear configuration;

FIG. 6 is a schematic view of the illustrative elongate treatment core of FIG. 5, shown in a spiral configuration;

FIG. 7 is a schematic view of an illustrative ultrasound radiating member that includes a one dimensional array of ultrasound transducers;

FIG. 8 is a schematic view of an illustrative ultrasound radiating member that includes a two dimensional array of ultrasound transducers;

FIG. 9 is a schematic view of a distal portion of an illustrative ultrasonic catheter; and

FIG. 10 is a schematic view of a distal portion of an illustrative ultrasonic catheter.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

As used herein, the term “ultrasonic energy” is used broadly, includes its ordinary meaning, and further includes mechanical energy transferred through pressure or compression waves with a frequency greater than about 20 kHz. Ultrasonic energy waves have a frequency between about 500 kHz and about 20 MHz in one example embodiment, between about 1 MHZ and about 3 MHz in another example embodiment, of about 3 MHz in another example embodiment, and of about 2 MHz in another example embodiment. As used herein, the term “catheter” is used broadly, includes its ordinary meaning, and further includes an elongate flexible tube configured to be inserted into the body of a patient, such as into a body part, cavity, duct or vessel. As used herein, the term “therapeutic compound” is used broadly, includes its ordinary meaning, and encompasses drugs, medicaments, dissolution compounds, genetic materials, microbubbles, nanobubbles, nanoparticles or phase-shift nanodroplets, and other substances capable of effecting physiological functions through either chemical reaction with substances within the body or through physical interaction with tissue in the body. A mixture comprising such substances is encompassed within this definition of “therapeutic compound”. As used herein, the term “end” is used broadly, includes its ordinary meaning, and further encompasses a region generally, such that “proximal end” includes “proximal region”, and “distal end” includes “distal region”.

As expounded herein, ultrasonic energy is often used to enhance the delivery and/or effect of a therapeutic compound. For example, in the context of treating vascular occlusions, ultrasonic energy has been shown to increase enzyme mediated thrombolysis by enhancing the delivery of thrombolytic agents into a thrombus, where such agents lyse the thrombus by degrading the fibrin that forms the thrombus. The thrombolytic activity of the agent is enhanced in the presence of ultrasonic energy in the thrombus. However, it should be appreciated that the invention should not be limited to the mechanism by which the ultrasound enhances treatment unless otherwise stated. In other applications, ultrasonic energy has also been shown to enhance transfection of gene-based drugs into cells, and augment transfer of chemotherapeutic drugs into tumor cells. Ultrasonic energy delivered from within a patient's body has been found to be capable of producing non-thermal effects that increase biological tissue permeability to therapeutic compounds by up to or greater than an order of magnitude.

Use of an ultrasound catheter to deliver ultrasonic energy and a therapeutic compound directly to the treatment site mediates or overcomes many of the disadvantages associated with systemic drug delivery, such as low efficiency, high therapeutic compound use rates, and significant side effects caused by high doses. Local therapeutic compound delivery has been found to be particularly advantageous in the context of thrombolytic therapy, chemotherapy, radiation therapy, and gene therapy, as well as in applications calling for the delivery of proteins and/or therapeutic humanized antibodies. However, it should be appreciated that in certain arrangements the ultrasound catheter can also be used in combination with systemic drug delivery instead or in addition to local drug delivery. In addition, local drug delivery can be accomplished through the use of a separate device (e.g., catheter).

As will be described below, the ultrasound catheter can include two or more ultrasound radiating members positioned therein. Such ultrasound radiating members can include a transducer (e.g., a PZT transducer), which is configured to convert electrical energy into ultrasonic energy. In such embodiments, the PZT transducer is excited by specific electrical parameters (herein “power parameters” that cause it to vibrate in a way that generates ultrasonic energy).

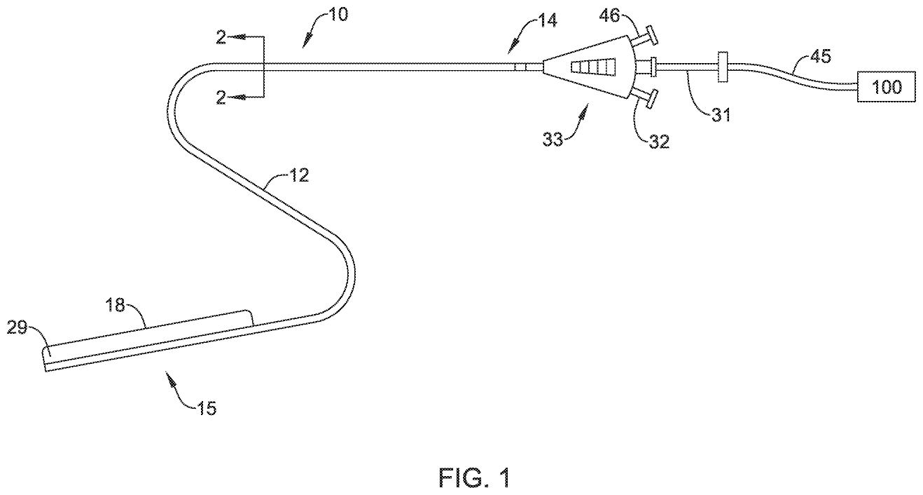

With reference to the illustrated embodiments, FIG. 1 illustrates an ultrasonic catheter 10 configured for use in a patient's vasculature. For example, in certain applications the ultrasonic catheter 10 is used to treat long segment peripheral arterial occlusions, such as those in the vascular system of the leg, while in other applications the ultrasonic catheter 10 is used to treat occlusions in the small vessels of the neurovasculature or other portions of the body (e.g., other distal portions of the vascular system). The ultrasonic catheter 10 may be used to treat PE (pulmonary embolisms) and DVT (deep vein thrombosis), for example. Thus, the dimensions of the catheter 10 may be adjusted based on the particular application for which the catheter 10 is to be used.

The ultrasonic catheter 10 generally includes a multi-component, elongate flexible tubular body 12 having a proximal region 14 and a distal region 15. The tubular body 12 includes a flexible energy delivery section 18 located in the distal region 15 of the catheter 10. The tubular body 12 and other components of the catheter 10 are manufactured in accordance with a variety of techniques. Suitable materials and dimensions are selected based on the natural and anatomical dimensions of the treatment site and on the desired percutaneous access site.

For example, in an embodiment the proximal region 14 of the tubular body 12 may include a material that has sufficient flexibility, kink resistance, rigidity and structural support to push the energy delivery section 18 through the patient's vasculature to a treatment site. Examples of such materials include, but are not limited to, extruded polytetrafluoroethylene (“PTFE”), polyethylenes (“PE”), polyamides and other similar materials. In certain embodiments, the proximal region 14 of the tubular body 12 may be reinforced by braiding, mesh or other constructions to provide increased kink resistance and pushability. For example, in certain embodiments nickel titanium or stainless steel wires may be placed along or incorporated into the tubular body 12 to reduce kinking.

In some instances, the energy delivery section 18 of the tubular body 12 may be formed of a material that (a) is thinner than the material forming the proximal region 14 of the tubular body 12, or (b) has a greater acoustic transparency than the material forming the proximal region 14 of the tubular body 12. Thinner materials generally have greater acoustic transparency than thicker materials. Suitable materials for the energy delivery section 18 include, but are not limited to, high or low density polyethylenes, urethanes, nylons, and the like. In some embodiments, the energy delivery section 18 is formed from the same material or a material of the same thickness as the proximal region 14.

One or more fluid delivery lumens may be incorporated into the tubular body 12. For example, in one embodiment a central lumen passes through the tubular body 12. The central lumen extends through the length of the tubular body 12, and is coupled to a distal exit port 29 and a proximal access port 31. The proximal access port 31 forms part of a hub 33, which is attached to the proximal region 14 of the catheter 10. In some cases, the hub 33 may include a cooling fluid fitting 46, which is hydraulically connected to a lumen within the tubular body 12. In some cases, the hub 33 may also include a therapeutic compound inlet port 32, which is hydraulically connected to a lumen within the tubular body 12. In some cases, the therapeutic compound inlet port 32 may also be hydraulically coupled to a source of therapeutic compound via a hub such as a Luer fitting.

The catheter 10 is configured to have two or more ultrasound radiating members positioned therein. For example, in certain embodiments an ultrasound radiating member may be fixed within the energy delivery section 18 of the tubular body, while in other embodiments a plurality of ultrasound radiating members are fixed to an assembly that is passed into the central lumen. In either case, the one or more ultrasound radiating members are electrically coupled to a control system 100 via a cable 45. In one embodiment, the outer surface of the energy delivery section 18 can include a cavitation promoting surface configured to enhance/promote cavitation at the treatment site. In some cases, a cavitation promoting surface is a textured surface that can retain small pockets of air when submerged. The small pockets of air can server as a source for microbubbles or nanobubbles, thereby reducing the threshold for cavitation in an ultrasound field. In some cases, the outer surface of the energy delivery section 18 may be coated with a coating that includes components that will lower the cavitation threshold. As an example, the surface may be hydrophobic and textured in a way so that the textured surface presents a lower cavitation threshold than the surrounding bulk fluid. This can enhance the therapeutic effect of the ultrasound.

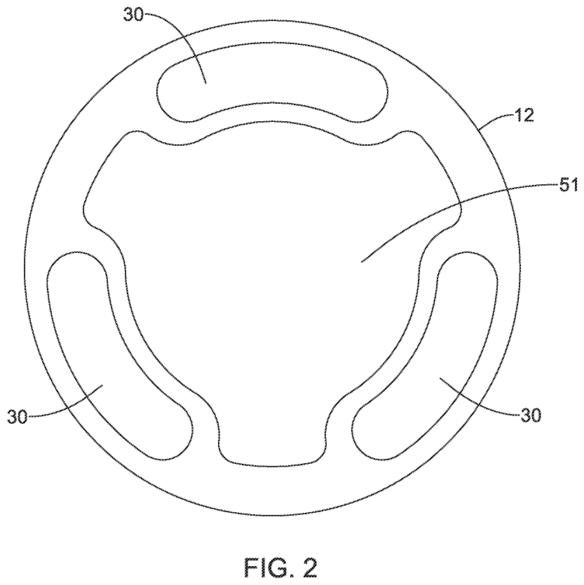

FIG. 2 illustrates a cross section of the tubular body 12 taken along line 2-2 of FIG. 1. As shown in FIG. 2, three fluid delivery lumens 30 may be incorporated into the tubular body 12. In other embodiments, more or fewer fluid delivery lumens can be incorporated into the tubular body 12. The tubular body 12 may include a hollow central lumen 51 passing through the tubular body 12. The cross-section of the tubular body 12, as illustrated in FIG. 2, may be substantially constant along most of the length of the catheter 10. Thus, in such embodiments, substantially the same cross-section is present in both the proximal region 14 and the distal region 15 of the catheter 10. In some cases, the cross-section may vary within the energy delivery section 18. In some cases, the central lumen 51 may be adapted to accommodate a coolant provided through the central lumen, even when a treatment core is present within the central lumen 51.

In certain embodiments, the central lumen 51 has a minimum diameter greater than about 0.030 inches. In another embodiment, the central lumen 51 has a minimum diameter greater than about 0.037 inches. In another embodiment, the fluid delivery lumens 30 have dimensions of about 0.026 inches wide by about 0.0075 inches high, although other dimensions may be used in other applications.

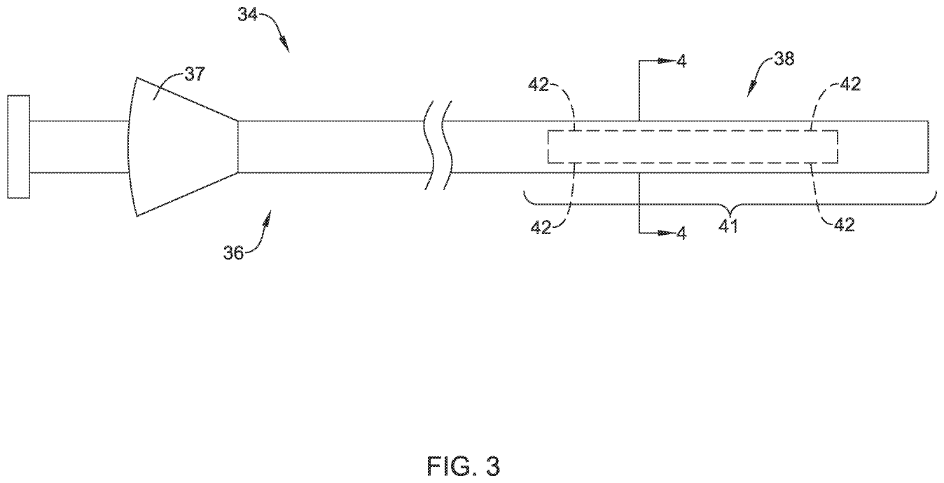

As described above, the central lumen 51 may extend through the length of the tubular body 12. As shown in FIG. 1, the central lumen 51 includes a distal exit port 29 and a proximal access port 31. The proximal access port 31 forms part of the hub 33, which is attached to the proximal region 14 of the catheter 10. The central lumen 51 may be configured to receive an elongate inner core 34 of which an embodiment is illustrated in FIG. 3. In some cases, the elongate inner core 34 includes a proximal region 36 and a distal region 38. A proximal hub 37 is fitted on the inner core 34 at one end of the proximal region 36. One or more ultrasound radiating members are positioned within an inner core energy delivery section 41 located within the distal region 38. The ultrasound radiating members form an ultrasound assembly 42, which will be described in detail below.

As used herein, the terms “ultrasonic energy”, “ultrasound” and “ultrasonic” are broad terms, having their ordinary meanings, and further refer to, without limitation, mechanical energy transferred through longitudinal pressure or compression waves. Ultrasonic energy can be emitted as continuous or pulsed waves, depending on the requirements of a particular application. Additionally, ultrasonic energy can be emitted in waveforms having various shapes, such as sinusoidal waves, triangle waves, square waves, or other wave forms. Ultrasonic energy includes sound waves. In certain embodiments, the ultrasonic energy has a frequency between about 20 kHz and about 20 MHz. For example, in one embodiment, the waves have a frequency between about 500 kHz and about 20 MHZ. In another embodiment, the waves have a frequency between about 1 MHz and about 3 MHz. In yet another embodiment, the waves have a frequency of about 2 MHz. The average acoustic power for each ultrasound radiating member is between about 0.01 watts and 300 watts. In some embodiments, the average acoustic power for each ultrasound radiating member is about 0.2 watts and about 2.5 watts. In an embodiment, the average acoustic power for each ultrasound radiating member is about 0.27 watts.

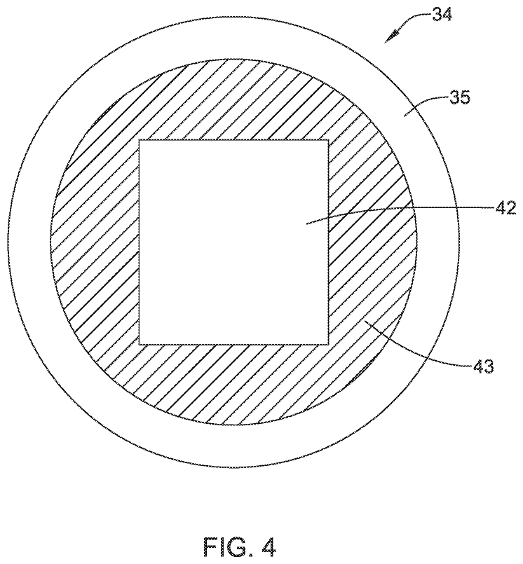

As shown in the cross-section illustrated in FIG. 4, which is taken along the line 4-4 of FIG. 3, the inner core 34 may have a cylindrical shape, with an outer diameter that permits the inner core 34 to be inserted into the central lumen 51 of the tubular body 12 via the proximal access port 31. Suitable outer diameters of the inner core 34 include, but are not limited to, about 0.010 inches to about 0.100 inches. In another embodiment, the outer diameter of the inner core 34 is between about 0.020 inches and about 0.080 inches. In yet another embodiment, the inner core 34 has an outer diameter of about 0.035 inches.

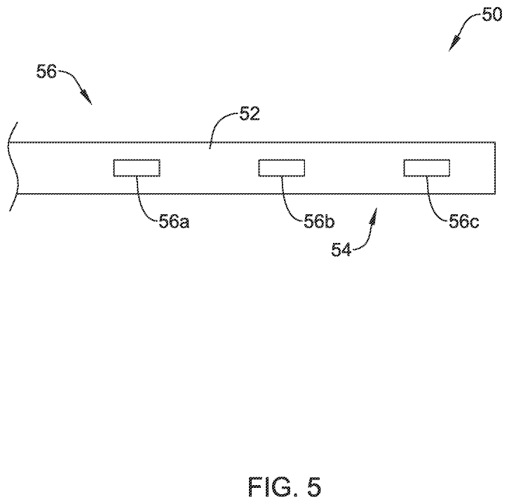

Still referring to FIG. 4, the inner core 34 may include a cylindrical outer body 35 that houses the ultrasound assembly 42. The ultrasound assembly 42 includes wiring and ultrasound radiating members, described in greater detail in FIGS. 5 through 7D, such that the ultrasound assembly 42 is capable of radiating ultrasonic energy from the energy delivery section 41 of the inner core 34. The ultrasound assembly 42 is electrically connected to the hub 33, where the inner core 34 can be connected to a control system 100 via cable 45 (illustrated in FIG. 1). In some cases, an electrically insulating potting material 43 fills the inner core 34, surrounding the ultrasound assembly 42, thus preventing movement of the ultrasound assembly 42 with respect to the outer body 35. In one embodiment, the thickness of the outer body 35 is between about 0.0002 inches and 0.010 inches. In another embodiment, the thickness of the outer body 35 is between about 0.0002 inches and 0.005 inches. In yet another embodiment, the thickness of the outer body 35 is about 0.0005 inches.

As used herein, the term “ultrasound radiating member” refers to any apparatus capable of producing ultrasonic energy. For example, in one embodiment, an ultrasound radiating member comprises an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics may include a crystalline material, such as quartz, that changes shape when an electrical voltage is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

FIG. 5 is a schematic view of an illustrative treatment core 50. In some cases, the illustrative treatment core 50 may be an ultrasound catheter, and may be considered as being an example of the elongate inner core 34 (FIG. 3). The treatment core 50 may be disposable within the flexible energy delivery section 18 of the tubular body 12. In some cases, the treatment core 50 may be considered as including an elongate shaft 52 that extends to a distal region 54. The elongate shaft 52 may be made of any suitable material, such as but not limited to polyimide, PET (polyethylene terephthalate) or epoxy, and may have a length in a range of 40 centimeters to 150 centimeters and a diameter in a range of 1 millimeter to 4 millimeter.

The treatment core 50 may include one or more ultrasound radiating members 56, individually labeled as 56a, 56b and 56c. It will be appreciated that only a small fraction of the elongate shaft 52 is visible in FIG. 5, and thus the treatment core 50 may include considerably more ultrasound radiating members 56. In some cases, the ultrasound radiating members 56 may be considered as having an axial spacing between adjacent ultrasound radiating members 56 that is in a range of 0.5 centimeters to 4 centimeters.

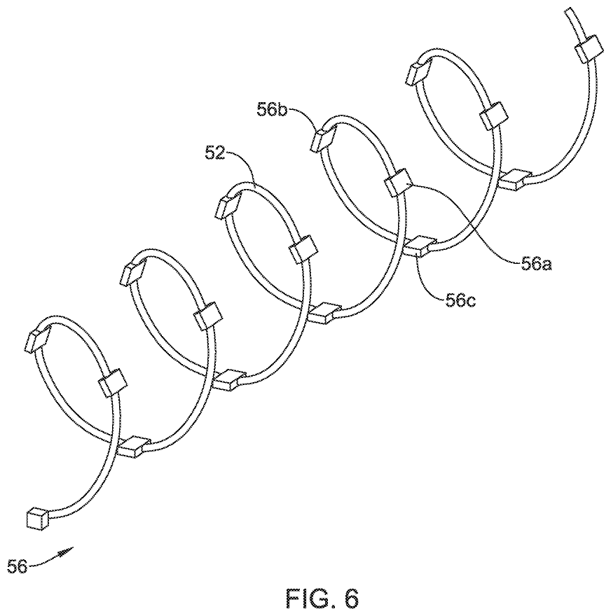

In some cases, the treatment core 50 may be adapted to move between a straight configuration (as seen in FIG. 5) and a helical configuration, as shown for example in FIG. 6. The helical configuration is just an example, as other configurations are contemplated. For example, an “S” shape or a “Z” shape may be useful for providing constructive interference. In some cases, the ultrasound radiating members 56 may be angled and/or shaped to provide constructive hindsight with a straight configuration.

FIG. 6 shows the treatment core 50 in a helical configuration. FIG. 6 shows that the treatment core 50 includes considerably more ultrasound radiating members 56 than the three that are schematically shown in FIG. 5. In some cases, the ultrasound radiating members 56 may be spaced 1 centimeters to 4 centimeters apart when the treatment core 50 is in the helical configuration.

In some cases, the ultrasound radiating members 56 may be arranged such that the treatment zone has a treatment zone length, when in the spiral configuration, that is in a range of 6 centimeters to 50 centimeters. In some cases, the treatment core 50 may have a spiral pitch when in the spiral configuration that is in a range of 3 centimeters to 6 centimeters. In some cases, the treatment core 50 may have a spiral diameter when in the spiral configuration that is in a range of 6 millimeters to 30 millimeters.

In some cases, the treatment core 50 may be adapted to have one or more properties such as but not limited to catheter size, spiral diameter, spiral pitch and treatment zone length (both linear and spiral) that may vary depending on the intended intravascular site that the treatment core 50 will be used at. For example, the treatment core 50 may vary depending on whether the disease state is a PAO (peripheral arterial occlusion) disease state, a DVT (deep vein thrombosis) disease state or a PE (pulmonary embolism) disease state. PAO is a disease state in which arteries have slowly closed with plaque over a long period of time, and then suddenly become completely occluded as a result of a clot forming in the narrowed vessel.

For a PAO disease state, the tubular body 12 may correspond to a 6 French (F) catheter. The spiral diameter may be 6 millimeters to 8 millimeters, the spiral pitch may be 3 centimeters to 6 centimeters, and the treatment zone (where the ultrasound radiating members 56 are located) having a length of 6 centimeters to 50 centimeters when spiraled and a length of 6 centimeters to 70 centimeters when linear. As a PAO-specific example, the spiral diameter may be 3 centimeters, the pitch may be 3 centimeters, the spiraled treatment zone length may be 20 centimeters, with about 6.7 spirals, and the straight treatment zone length may be about 66 centimeters. The transducer alignment may be 120 degrees, and the linear spacing between transducers may be about 3.3 centimeters.

For a DVT disease state, the tubular body 12 may correspond to an 8F (or larger) catheter. The spiral diameter may be 8 millimeters to 16 millimeters, the spiral pitch may be 3 centimeters to 6 centimeters, and the treatment zone (where the ultrasound radiating members 56 are located) having a length of 6 centimeters to 50 centimeters when spiraled, and a length of 6 centimeters to 100 centimeters when linear. As a DVT-specific example, the spiral diameter may be 1.6 centimeters, the pitch may be 6 centimeters, the spiraled treatment zone length may be 30 centimeters, with about 5 spirals, and the straight treatment zone length may be about 39 centimeters. The transducer alignment may be 120 degrees, and the linear spacing between transducers may be about 2.6 centimeters.

For a PE disease state, the tubular body 12 may correspond to an 8F (or larger) catheter. The spiral diameter may be 16 millimeters to 30 millimeters, the spiral pitch may be 3 centimeters to 6 centimeters, and the treatment zone (where the ultrasound radiating members 56 are located) having a length of 6 centimeters to 20 centimeters when spiraled and a length of 6 centimeters to 70 centimeters when linear. As a PE-specific example, the spiral diameter may be 0.8 centimeters, the pitch may be 3.33 centimeters, the spiraled treatment zone length may be 30 centimeters, with about 9 spirals. The transducer alignment may be 120 degrees, and the linear spacing between transducers may be about 1.4 centimeters.

The treatment core 50 may be designed to self-spiral automatically as it exits from a guide catheter in the treatment area of interest. Alternatively, the spiral action may be mechanically controlled by the physician after the treatment core 50 is placed in the treatment area in its straight configuration. The mechanical control could control both the spiral diameter and the pitch distance or may control only one or the other of those spiral parameters. In some cases, the elongate shaft 12 may be adapted to spiral with the treatment core 50 disposed within the elongate shaft 12. In some cases, the treatment core 50 may include one or more additional polymeric layers that provide the treatment core 50 with fluid delivery lumens that replace the fluid delivery lumens 30 that extend within the elongate shaft 12.

In some cases, the ultrasound radiating members 56 may be operated in a pulsed mode. For example, the time average electrical power supplied to each of the ultrasound radiating members 56 may be between about 0.001 watts and about 5 watts and may be between about 0.05 watts and about 3 watts. In some embodiments, the time average electrical power over treatment time may be about 0.45 watts or 1.2 watts. The duty cycle may be between about 0.01% and about 90% and may be between about 0.1% and about 50%. In some embodiments, the duty ratio may be about 7.5%, 15% or a variation between 1% and 30%. The pulse average electrical power may be between about 0.01 watts and about 40 watts and may be between about 0.1 watts and 20 watts. In some embodiments, the pulse averaged electrical power may be about 4 watts, 8 watts, 16 watts, or a variation of 1 to 16 watts. The amplitude, pulse width, pulse repetition frequency, average acoustic pressure or any combination of these parameters may be constant or varied during each pulse or over a set of pulses. In a non-linear application of acoustic parameters the above ranges can change significantly. Accordingly, the overall time average electrical power over treatment time may stay constant throughout the treatment or may vary in real time during the treatment. the same but not real-time average power.

In some cases, the pulse repetition may be between about 1 Hz and about 2 kHz and more can be between about 1 Hz and about 50 Hz. In some embodiments, the pulse repetition rate may be about 30 Hz, or a variation of about 10 Hz to about 40 Hz. The pulse duration or width may be between about 0.5 millisecond and about 50 milliseconds and may be between about 0.1 millisecond and about 25 milliseconds. In some embodiments, the pulse duration may be about 2.5 milliseconds, 5 milliseconds or a variation of 1 to 18 milliseconds. In addition, the average acoustic pressure may be between about 0.1 to about 4 MPa or in between about 0.5 or about 0.74 to about 3.7 MPa.

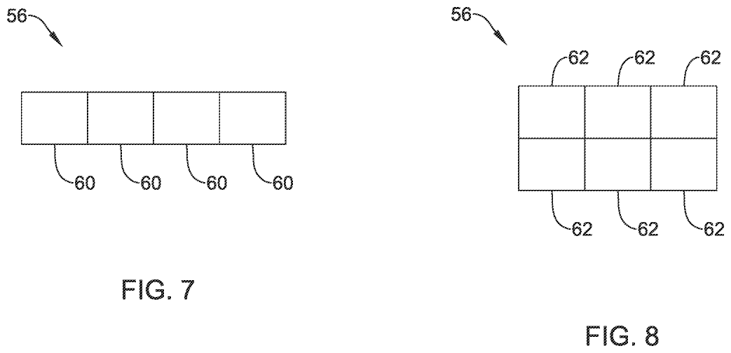

In some cases, each of the ultrasound radiating members 56 may be piezoelectric material and may be of various shapes. The ultrasound radiating members 56 may be cylindrical or rectangular or disc shaped. Each ultrasound radiating member 56 may be a single transducer or they may be designed as an array of small transducers. FIG. 7 schematically shows an ultrasound radiating member 56 that is designed as a one-dimensional array including (as shown) a total of four ultrasound transducers 60. The ultrasound radiating member 56 may include any number of ultrasound transducers 60 arranged in a one-dimensional array. FIG. 8 schematically shows an ultrasound radiating member 56 that is designed as a two-dimensional array including (as shown) a total of six ultrasound transducers 62 arranged in a two-dimensional array having two rows of three ultrasound transducers 62 each.

The ultrasound transducers 60 and 62 may be adapted to radiate ultrasound energy radially in multiple directions from the ultrasound transducers 60 and 62 or to radiate preferentially in one radial direction. The position of the ultrasound transducers 60 and 62 and the directions of ultrasound radiation may be arranged so that at some of the radiated ultrasound field will overlap with radiated ultrasound fields from ultrasound transducers 60 and 62 that are proximal or distal in the spiral treatment core 50. The ultrasound radiating members 56 may be unfocused transducers or may be designed as focused transducers in order to increase the ultrasound intensity in certain beam directions and at a certain distance from the transducers. The ultrasound radiating members 56 may include two transducers which can be operated at two different frequencies at the same time while radiating ultrasound energy into the same or overlapping volumes. In some cases, the ultrasound fields emitted by neighboring ultrasound transducers may be adapted to provide constructive interference.

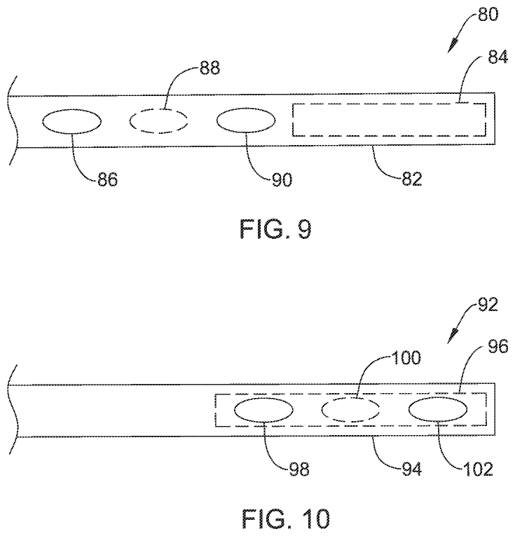

The ultrasonic catheter 10 (or the treatment core 50) may be used to deliver a therapeutic or diagnostic substance into a treatment zone. FIG. 9 is a schematic view of a distal portion of an illustrative ultrasound catheter 80 that may be considered as an example of the ultrasonic catheter 10. The ultrasound catheter 80 includes an elongate shaft 82. An ultrasound assembly 84 is disposed within the elongate shaft 82. In some cases, the ultrasound assembly 84, which may include one or more distinct ultrasound radiating members, may be secured within the elongate shaft 82. In some cases, the ultrasound assembly 84 may be part of a treatment core that is slidingly disposable within a lumen extending through the elongate shaft 82.

The ultrasound catheter 80 includes several drug elution ports that allow a therapeutic or diagnostic substance to be carried through the elongate shaft 82 and to be eluted near a treatment zone. As shown, the ultrasound catheter 80 includes a first drug elution port 86, a second drug elution port 88 shown in phantom because it is on the back side (in the illustrated orientation) of the elongate shaft 82, and a third drug elution port 90. In some cases, as shown, the drug elution ports 86, 88 and 90 are oval, and may be considered as being adapted to permit microbubbles, nanobubbles, nanoparticles or phase-shift droplets to exit the elongate shaft 82.

While a total of three drug elution ports 86, 88 and 90 are shown, it will be appreciated that this is merely illustrative, as the ultrasound catheter 80 may include any number of drug elution ports. While not shown, it will be appreciated that the elongate shaft 82 includes one or more fluid lumen that are fluidly coupled with the drug elution ports 86, 88 and 90. In some cases, a single fluid lumen may be fluidly coupled with all of the drug elution ports 86, 88 and 90. In some cases, each of the drug elution ports 86, 88 and 90 may be fluidly coupled with a separate fluid lumen.

In some cases, as shown, all of the drug elution ports 86, 88 and 90 are proximal of where the ultrasound assembly 84 is located within the ultrasound catheter 80. In some cases, this may allow therapeutic or diagnostic substances to be delivered upstream of a treatment area, and allow blood flow to carry the therapeutic or diagnostic substances into the treatment area. As an example, the drug may be delivered 0 to 10 centimeters upstream of the treatment area.

In some cases, as shown in FIG. 10, the drug elution ports may be located closer to the treatment area. FIG. 10 shows an ultrasound catheter 92 that may be considered as an example of the ultrasonic catheter 10. The ultrasound catheter 92 includes an elongate shaft 94. An ultrasound assembly 96 is disposed within the elongate shaft 94. In some cases, the ultrasound assembly 96, which may include one or more distinct ultrasound radiating members, may be secured within the elongate shaft 94. In some cases, the ultrasound assembly 96 may be part of a treatment core that is slidingly disposable within a lumen extending through the elongate shaft 94.

The ultrasound catheter 92 includes several drug elution ports that allow a therapeutic or diagnostic substance to be carried through the elongate shaft 94 and to be eluted near a treatment zone. As shown, the ultrasound catheter 92 includes a first drug elution port 98, a second drug elution port 100 shown in phantom because it is on the back side (in the illustrated orientation) of the elongate shaft 94, and a third drug elution port 102. In some cases, as shown, the drug elution ports 98, 100 and 102 are oval, and may be considered as being adapted to permit microbubbles, nanobubbles, nanoparticles or phase-shift nanodroplets to exit the elongate shaft 94.

While a total of three drug elution ports 98, 100 and 102 are shown, it will be appreciated that this is merely illustrative, as the ultrasound catheter 92 may include any number of drug elution ports. While not shown, it will be appreciated that the elongate shaft 94 includes one or more fluid lumen that are fluidly coupled with the drug elution ports 98, 100 and 102. In some cases, a single fluid lumen may be fluidly coupled with all of the drug elution ports 98, 100 and 102. In some cases, each of the drug elution ports 98, 100 and 102 may be fluidly coupled with a separate fluid lumen.

As can be seen in FIG. 10, the drug elution ports 98, 100 and 102 overlap where the ultrasound assembly 96 is located. As a result, the therapeutic or diagnostic substances may be delivered directly within a treatment area. The drug delivery lumens and ports may be designed to deliver drug in a substantially even manner along the treatment zone or may be designed to deliver drug preferentially to one area or another of the treatment zone. The drug delivery ports may be designed to deliver drug into the center of the spiral or towards the vessel wall away from the center of the spiral.

In some cases, the size of the drug delivery lumens and drug delivery ports may be large enough to allow adequate flow at low pressures (<5 mmHg). The drug delivery lumens and drug delivery ports will be designed to accommodate ultrasound contrast microbubbles as well as non-solution substances such as perfluorocarbon nanodroplets (phase-shift nanodroplets) or drug loaded nanoparticles.

The materials that can be used for the various components of the devices described herein may include those commonly associated with medical devices. The devices and components thereof described herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the devices described herein to achieve the same result.

In some embodiments, a degree of Magnetic Resonance Imaging (MRI) compatibility is imparted into the devices described herein. For example, the devices described herein, or portions thereof, may be made of a material that does not substantially distort the image and create substantial artifacts (e.g., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for treating a vascular region, the system comprising:
an elongate catheter shaft having a distal end region, the elongate catheter shaft defining an inner lumen extending through the elongate catheter shaft and a fluid delivery lumen extending adjacent to the inner lumen; and
a treatment core disposed within the inner lumen, the treatment core including a plurality of ultrasound transducers that are adapted to provide constructive interference.

2. The system of claim 1, wherein the elongate catheter shaft is adapted to move between a straight configuration and a helical configuration.

3. The system of claim 1, wherein the treatment core is adapted to move between a straight configuration and a helical configuration.

4. The system of claim 1, wherein the fluid delivery lumen has a distal end that is proximal of any of the plurality of ultrasound transducers.

5. The system of claim 1, wherein the fluid delivery lumen comprises a plurality of fluid delivery lumens, and at least some of the plurality of fluid delivery lumens has a distal end that is offset from a distal end of others of the plurality of fluid delivery lumens.

6. The system of claim 1, wherein the plurality of ultrasound transducers has a treatment zone length when in the spiral configuration in a range of 6 centimeters to 50 centimeters.

7. The system of claim 1, wherein the plurality of ultrasound transducers has a spiral pitch when in the spiral configuration that is in a range of 3 centimeters to 6 centimeters.

8. The system of claim 1, wherein the plurality of ultrasound transducers has a spiral diameter when in the spiral configuration that is in a range of 6 millimeters to 30 millimeters.

9. The system of claim 1, wherein each of the plurality of ultrasound transducers are spaced 1 centimeter to 4 centimeters from adjacent ultrasound transducers when the plurality of ultrasound transducers are in the spiral configuration.

10. An ultrasonic catheter system, comprising:
a multi-lumen catheter shaft having a central lumen and a plurality of fluid delivery lumens; and
an ultrasound catheter core disposed within the central lumen, the ultrasound catheter core including a plurality of ultrasound transducers adapted to move between a straight configuration and a helical configuration;
wherein the plurality of ultrasound transducers are adapted to provide constructive interference when in the helical configuration; and
wherein the central lumen is adapted to allow for a cooling media to pass therethrough when the ultrasound catheter core is disposed within the central lumen.

11. The ultrasonic catheter system of claim 10, wherein the multi-lumen catheter shaft is adapted to move between a straight configuration and a helical configuration.

12. The ultrasonic catheter system of claim 10, wherein the ultrasound catheter core is adapted to move between a straight configuration and a helical configuration.

13. The ultrasonic catheter system of claim 10, wherein at least some of the plurality of fluid delivery lumens has a distal end that is offset from a distal end of others of the plurality of fluid delivery lumens.

14. The ultrasonic catheter system of claim 10, wherein the plurality of ultrasonic transducers are arranged for treating a PAO (peripheral arterial occlusion) disease state.

15. The ultrasonic catheter system of claim 10, wherein the plurality of ultrasonic transducers are arranged for treating DVT (deep vein thrombosis).

16. The ultrasonic catheter system of claim 10, wherein the plurality of ultrasonic transducers are arranged for treating PE (pulmonary embolisms).

17. A system for treating a vascular region, the system comprising:
an elongate catheter shaft having a distal end region, the elongate catheter shaft defining an inner lumen extending through the elongate catheter shaft and a fluid delivery lumen extending adjacent to the inner lumen; and
a treatment core disposed within the inner lumen, the treatment core including two or more ultrasound radiating members, each of the two or more ultrasound radiating members adapted to provide constructive interference when in the helical configuration.

18. The system of claim 17, wherein each of the two or more ultrasound radiating members comprise a one-dimensional array of ultrasound transducers.

19. The system of claim 17, wherein each of the two or more ultrasound radiating members comprise a two-dimensional array of ultrasound transducers.

20. The system of claim 17, wherein one of the two or more ultrasound radiating members emit a radiated ultrasound field that overlaps with the radiated ultrasound field emitted by another of the two or more ultrasound radiating members.
